(19) United States
(12) Patent Application Publication
(10) Pub. No.: US 2019/0192004 A1
(43) Pub. Date: Jun. 27, 2019

(54) BIOLOGICAL INFORMATION MEASURING APPARATUS AND BIOLOGICAL INFORMATION DETECTION SENSOR

(71) Applicant: SEIKO EPSON CORPORATION, Tokyo (JP)

(72) Inventors: Atsushi MATSUO, Azumino-shi (JP); Yoshiaki MATSUMOTO, Ina-shi (JP)

(73) Assignee: SEIKO EPSON CORPORATION, Tokyo (JP)

(21) Appl. No.: 16/228,959

(22) Filed: Dec. 21, 2018

(30) Foreign Application Priority Data
Dec. 25, 2017 (JP) 2017-247754

(51) Int. Cl. A61B 5/00

(52) U.S. Cl. CPC A61B 5/0059; A61B 5/02108

(57) ABSTRACT

A biological information measuring apparatus includes a housing having a contact part to be in contact with a body of a user, a light emitter that emits light toward the body of the user, a light receiver that receives a reflected light reflected by the body of the user, a processing unit that processes a signal from the light receiver and determines biological information, and a optically transparent member placed in the contact part and transmitting the light emitted by the light emitter and the light entered into the light receiver, and the light emitter and the light receiver are placed on the optically transparent member.

### CROSS-REFERENCES TO RELATED APPLICATIONS

This application claims priority to Japanese Patent Application No. 2017-247754, filed Dec. 25, 2017, the entirety of which is herein incorporated by reference.

### BACKGROUND

### 1. Technical Field

The present invention relates to a biological information measuring apparatus and biological information detection sensor.

### 2. Related Art

In related art, measuring apparatuses that can measure biological information including pulse wave of living bodies. As the measuring apparatus, a measuring apparatus including a sensor having a light emitting part that emits light to a living body and a light receiving part that receives the light reflected by the living body, a substrate on which the sensor is mounted, a contact part that suppresses direct contact of the sensor with the living body, and a partition member is known (for example, Patent Document 1 (JP-A-2017-148139)).

In the measuring apparatus disclosed in Patent Document 1, the partition member suppresses reception of other light (e.g. reflected light from the living body surface) than the light from inside of the living body when the light receiving part receives the light from inside of the living body. The partition member has a sensor contact surface with an area to be in contact with the upper surface of the sensor, a living body contact surface to be in contact with the living body at measurement, and a light shielding surface that shields passing of the light.

Here, when the distance from the living body to the light receiving part is longer for the reflected light, there is a problem that the reflected light is attenuated and the amount of light received by the light receiving part is smaller. For example, in the measuring apparatus disclosed in Patent Document 1, the living body contact surface in the partition part in which the sensor contact surface is in contact with the upper surface of the sensor comes into contact with the living body at the measurement of biological information, and the distance tends to be longer and the amount of light received by the light receiving part tends to be smaller. When the amount of light received by the light receiving part becomes smaller, another problem that detection accuracy of the biological information becomes lower arises.

### SUMMARY

An advantage of some aspects of the invention is to provide a biological information measuring apparatus and biological information detection sensor in which detection accuracy of biological information may improved.

A biological information measuring apparatus according to a first aspect of the invention includes a housing having a contact part to be in contact with a body of a user, a light emitting part that emits light toward the body of the user, a light receiving part that receives reflected light reflected by the body of the user, a processing unit that processes a signal from the light receiving part and determines biological information, and a optically transparent member placed in the contact part and transmitting the light emitted by the light emitting part and the light to be entered into the light receiving part, wherein the light emitting part and the light receiving part are placed on the optically transparent member.

According to the configuration, the light emitting part and the light receiving part are placed on the optically transparent member provided in the contact part to be in contact with the body of the user in the housing and transmitting the light emitted by the light emitting part and the light to be entered into the light receiving part. Thereby, compared to the configuration of Patent Document 1, the distances between the optically transparent member and the light emitting part and light receiving part can be made shorter. Accordingly, attenuation of the light emitted from the light emitting part to the body of the user and attenuation of the light reflected by the body of the user and received by the light receiving part can be suppressed. Therefore, detection and measurement accuracy of the biological information by the biological information measuring apparatus can be improved and precision of the measured biological information can be improved.

Further, the distances between the optically transparent member and the light emitting part and light receiving part can be made shorter, and thereby, the biological information measuring apparatus can be made thinner.

In the first aspect, it is preferable that the optically transparent member has a first surface that can be in contact with the body of the user, and a second surface opposed to the first surface, on which the light emitting part and the light receiving part are placed.

Note that the second surface opposed to the first surface is an opposite surface to the first surface. Further, the optically transparent member may be a plate-like member and the plate-like member includes a member with at least one of the first surface and the second surface formed in a curved surface shape.

According to the configuration, the light emitting part and the light receiving part are placed on the second surface opposed to the first surface to be in contact with the user in the optically transparent member. Thereby, exposure of the light emitting part and the light receiving part outside of the housing can be reliably suppressed. Therefore, the light emitting part and the light receiving part can be protected.

Further, the first surface can be in contact with the body of the user, and thereby, the distance between the optically transparent member and the body of the user can be made shorter. Accordingly, the optical path length of the light reaching the body of the user from the light emitting part and the optical path length of the light reaching the light receiving part from the body of the user can be made shorter, and the above described attenuation of the lights can be further suppressed. Therefore, the measurement accuracy of the biological information by the biological information measuring apparatus can be further improved, and the precision of the measured biological information can be further improved.

In the first aspect, it is preferable that the optically transparent member has a convex portion projecting in a direction from the second surface toward the first surface and overlapping with the light emitting part as seen along a direction from the first surface toward the second surface.

According to the configuration, the convex portion overlapping with the light emitting part can be allowed to function as a convex lens that acts on the light emitted from the light emitting part. Accordingly, the light emitted from the light emitting part can be diffused by the lens effect of the convex portion. Therefore, the light emitted from the light emitting part can be efficiently output to the body of the user and the measurement accuracy of the biological information can be improved.

In the first aspect, it is preferable that the optically transparent member has a concave portion recessed in a direction from the first surface toward the second surface and overlapping with the light receiving part as seen along the direction from the first surface toward the second surface.

According to the configuration, the concave portion overlapping with the light receiving part can be allowed to function as a concave lens that acts on the light entered into the light receiving part. Accordingly, the light reflected by the body of the user and diffused can be collected in the light receiving part by the lens effect of the concave portion. Therefore, the light can be efficiently entered into the light receiving part and the measurement accuracy of the biological information can be improved.

In the first aspect, it is preferable that a connecting part that connects the processing unit and the second surface is provided, and the second surface has an electrode pattern that electrically connects the connecting part and the light emitting part and light receiving part.

Note that the electrode pattern can be formed on the second surface by e.g. evaporation or pattern printing of a conducting material.

According to the configuration, electric power can be supplied to the light emitting part and the detection signal can be transmitted from the light receiving part using the electrode pattern located on the second surface. Thereby, compared to the case where the power is supplied and the signal is transmitted to the light emitting part and the light receiving part via cables, the configuration of the biological information measuring apparatus can be simplified. Therefore, assembly of the sensor can be easily performed and the sensor can be made thinner.

Further, the light emitting part and light receiving part and the processing unit can be reliably connected via the connecting part connected to the electrode pattern.

In the first aspect, it is preferable that the light emitting part includes a light emitting device that emits light, and a reflection member that covers the light emitting device and has a concave curved surface for reflecting the light entered from the light emitting device toward the body of the user.

According to the configuration, the light generated in the light emitting device can be collected and output by the concave curved surface, and effectively radiated to the body of the user. Therefore, use efficiency of the light emitted from the light emitting device can be improved.

Further, the reflection member covers the light emitting device, and thereby, entry of part of the light emitted from the light emitting device into a component within the housing can be suppressed. Therefore, deterioration of components due to entry of unnecessary light can be suppressed.

In the first aspect, it is preferable that the optically transparent member is one of a glass substrate and a transparent resin.

According to the configuration, the glass substrate generally has higher strength and can easily maintain the shape of the optically transparent member even when the optically transparent member is in contact with the body of the user. Further, the glass substrate is generally inexpensive, and rise of the manufacturing cost of the biological information measuring apparatus can be suppressed.

Furthermore, the optically transparent member formed using a transparent resin can be manufactured to be relatively light, and thereby, weight increase of the biological information measuring apparatus can be suppressed, and additionally, the optically transparent member can be manufactured using the transparent resin mixed with coloring matter or the like and used as a filter.

In the first aspect, it is preferable that the optically transparent member has a shield area provided in a position except a passage area for the light emitted from the light emitting part and the light entering the light receiving part and shielding light.

According to the configuration, light is shielded by the shield area, and observation inside of the housing from outside can be suppressed. Therefore, the appearance of the biological information measuring apparatus can be made better.

A biological information detection sensor according to a second aspect of the invention is a biological information detection sensor used for a biological information measuring apparatus that measures biological information of a user, including a optically transparent member having a first surface that can be in contact with a body of the user, and a second surface as a surface opposed to the first surface, a light emitting part located on the second surface and emitting light from a side of the second surface to a side of the first surface, and a light receiving part located on the second surface and receiving light traveling from the side of the first surface to the side of the second surface, wherein the optically transparent member has a passage area through which at least the light emitted from the light emitting part and the light entering the light receiving part pass, and a shield area provided in a position except the passage area and shielding light.

Note that, as described above, the second surface opposed to the first surface is an opposite surface to the first surface. Further, the optically transparent member can be a plate-like member and the plate-like member includes a member with at least one of the first surface and the second surface formed in a curved surface shape.

According to the configuration, the same advantages as those of the biological information measuring apparatus can be offered. That is, in the optically transparent member, the light emitting part and the light receiving part are located on the second surface opposed to the first surface that can be in contact with the body of the user, and the distances between the first surface and the light emitting part and light receiving part can be made shorter. Accordingly, attenuation of the light emitted from the light emitting part to the body of the user and attenuation of the light reaching the light receiving part from the body of the user and received by the light receiving part can be suppressed. Therefore, detection accuracy of the biological information can be improved. Further, the distances between the first surface and the light emitting part and light receiving part (particularly, the distances between the optically transparent member and the light emitting part and light receiving part) can be made shorter, and thereby, the biological information detection sensor can be made thinner.

### BRIEF DESCRIPTION OF THE DRAWINGS

The invention will be described with reference to the accompanying drawings, wherein like numbers reference like elements.

FIG. 1 is a schematic diagram showing an example of use of a biological information measuring apparatus according to a first embodiment of the invention.

FIG. 2 shows an appearance of the biological information measuring apparatus in the first embodiment.

FIG. 3 is a rear view showing the biological information measuring apparatus in the first embodiment.

FIG. 4 is a block diagram showing a configuration of the biological information measuring apparatus in the first embodiment.

FIG. 5 shows a sensor in the first embodiment as seen from an opposite side to a light-exiting side.

FIG. 6 shows the sensor in the first embodiment as seen from a side.

FIG. 7 shows a sensor of a biological information measuring apparatus according to a second embodiment of the invention as seen from a light-exiting side.

FIG. 8 is a schematic diagram showing a sensor of a biological information measuring apparatus according to a third embodiment of the invention.

FIG. 9 is a schematic diagram showing optical paths of lights emitted from a light emitting part and optical paths of lights received by a light receiving part of the sensor in the third embodiment.

### DESCRIPTION OF EXEMPLARY EMBODIMENTS

### First Embodiment

As below, the first embodiment of the invention will be explained with reference to the drawings.

### General Configuration of Biological Information Measuring Apparatus

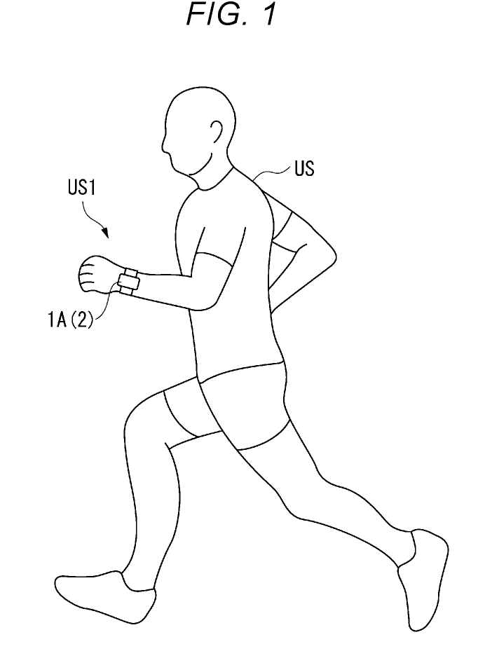

FIG. 1 is a schematic diagram showing an example of use of a biological information measuring apparatus 1A according to the embodiment.

As shown in FIG. 1, the biological information measuring apparatus 1A according to the embodiment (hereinafter, may be abbreviated as “measuring apparatus 1A”) is a wearable apparatus worn and used on a body of a user US and measuring biological information of the user US. Specifically, the measuring apparatus 1A is attached to an attached part US1 such as a wrist of the user US and used, and detects pulse wave of the user US as biological information and measures a pulse rate as biological information.

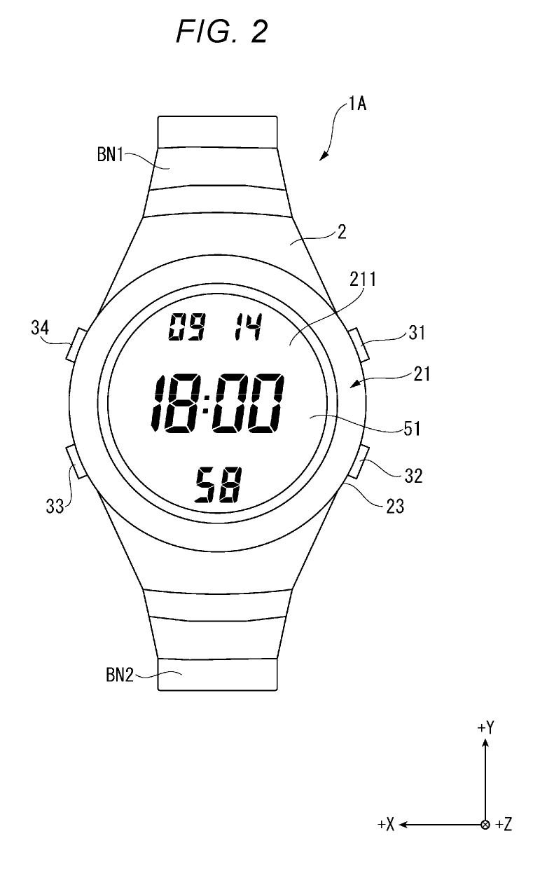

FIG. 2 shows an appearance of the measuring apparatus 1A.

As shown in FIG. 2, the measuring apparatus 1A includes a housing 2, and bands BN1 and BN2 provided on the housing 2.

Note that, in the subsequent drawings including FIG. 2, a direction from a front part 21 toward a back part 22 of the housing 2 is referred to as “+Z-direction”. Two directions orthogonal to the +Z-direction are referred to as “+X-direction” and “+Y-direction”, the +X-direction is the nine o'clock direction and the +Y-direction is the twelve o'clock direction as seen from the position facing the front part 21. Further, an opposite direction to the +Z-direction is referred to as “−Z-direction” (not shown). The same applies to “−X-direction” and “−Y-direction”.

The bands BN1 and BN2 are connected to end portions on sides in the ±Y-directions of the housing 2, and the band BN1 extends toward the side in the +Y-direction and the band BN2 extends toward the side in the −Y-direction with respect to the housing 2. These bands BN1 and BN2 are coupled to each other by a clasp (not shown), and thereby, the housing 2 is attached to the attached part US1. Note that the bands BN1 and BN2 may be integrally formed with the housing 2.

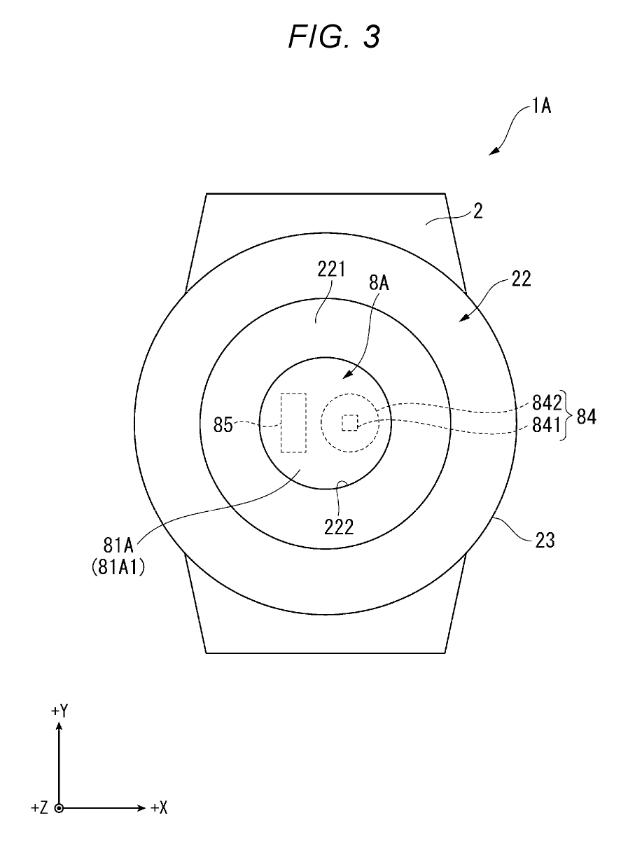

The housing 2 has the front part 21, a back part 22 (see FIG. 3), and a side part 23.

The front part 21 is a part located on the side in the −Z-direction in the housing 2 and can be visually recognized by the user US wearing the measuring apparatus 1A in the housing 2. A display part 51 forming an informing unit 5, which will be described later, is provided nearly at the center of the front part 21, and the display part 51 is covered by a circular cover 211.

The side part 23 is an annular part formed along the circumferential direction around the +Z-direction, and connects the front part 21 and the back part 22. Buttons 31, 32 forming an operation unit 3, which will be described later, are placed in a region on the side in the −X-direction in the side part 23, and buttons 33, 34 also forming the operation unit 3 are placed in a region on the side in the +X-direction. The buttons 31 to 34 are buttons projected and recessed relative to the housing 2.

FIG. 3 is a rear view showing the measuring apparatus 1A, specifically showing the back part 22 of the housing 2. Note that, in FIG. 3, the buttons 31 to 34 are not shown.

The back part 22 is a part located on the side in the +Z-direction in the housing 2 and contact part facing a contacted part of the user US and coming into contact with the contacted part (the body of the user US) in the housing 2.

A projecting portion 221 having a nearly annular shape is formed at the center of the back part 22. A circular opening portion 222 is formed at the center of the projecting portion 221. Inside of the opening portion 222, a light emitting part 84 and a light receiving part 85 forming a sensor 8A, which will be described later, are placed.

### Configuration of Housing

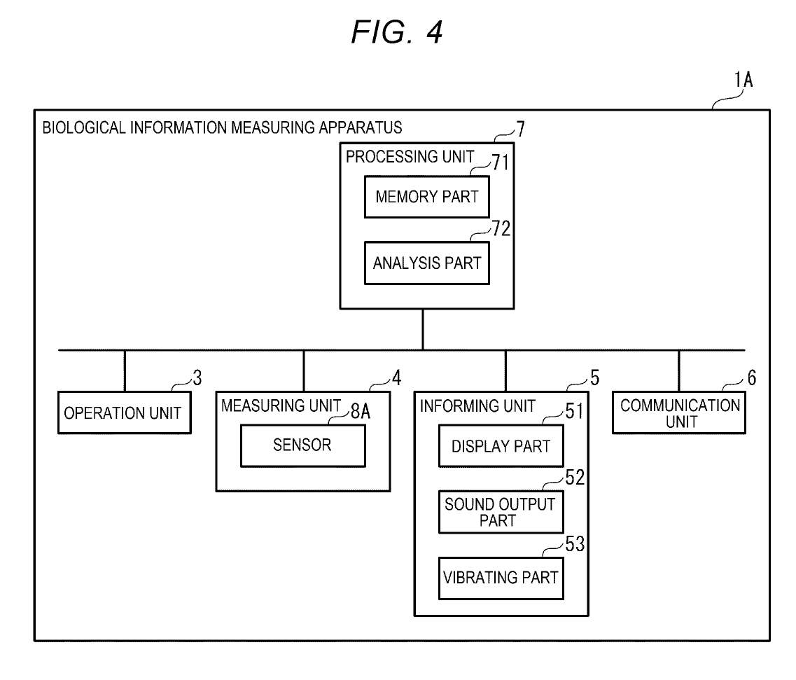

FIG. 4 is a block diagram showing a configuration of the measuring apparatus 1A.

As shown in FIG. 4, the measuring apparatus 1A has the operation unit 3, a measuring unit 4, the informing unit 5, a communication unit 6, and a processing unit 7 in addition to the housing 2, and these units are provided in the housing 2.

The operation unit 3 has the above described buttons 31 to 34 and outputs operation signals in response to input of the buttons 31 to 34 to the processing unit (processor) 7.

The measuring unit 4 detects biological information and outputs a detection result to the processing unit 7. The measuring unit 4 has the sensor 8A that detects the pulse wave as the biological information, and the sensor 8A outputs a pulse wave signal (detection signal) as the detection result to the processing unit 7. Note that the configuration of the sensor 8A will be described later in detail.

The informing unit 5 informs the user of various kinds of information under control by the processing unit 7. The informing unit 5 has the display part 51, a sound output part 52, and a vibrating part 53.

The display part 51 has various display panels of liquid crystal, electronic paper, etc., and displays information input from the processing unit 7. For example, the display part 51 displays the pulse rate detected and analyzed by the measuring unit 4.

The sound output part 52 outputs sound according to a sound signal input from the processing unit 7.

The vibrating part 53 has a motor that operates under control by the processing unit 7, and informs the user of e.g. a warning by vibration generated by driving the motor.

The communication unit 6 is a communication module that transmits the detected and measured biological information to an external apparatus, and further, outputs information received from the external apparatus to the processing unit 7. Note that, in the embodiment, the communication unit 6 wirelessly communicates with the external apparatus by the near field communication system, however, may communicate with the external apparatus via a relay device such as a cradle or cable. Alternatively, the communication unit 6 may communicate with the external apparatus via a network.

The processing unit 7 is a circuit board having an arithmetic processing circuit and a flash memory, and electrically connected to the operation unit 3, the measuring unit 4, the informing unit 5, and the communication unit 6. The processing unit 7 executes control processing of controlling the operation of the measuring apparatus 1A autonomously or according to the operation signal input from the operation unit 3. In addition, the processing unit 7 controls the operation of the measuring unit 4 (sensor 8A) to analyze the pulse wave signal indicating the pulse wave as a kind of biological information detected by the measuring unit 4 and determine the pulse rate as a kind of biological information.

The processing unit 7 has a memory part 71 formed by the flash memory, and an analysis part 72 formed by the arithmetic processing circuit that executes a program stored in the memory part 71.

The memory part 71 stores various programs and data necessary for the operation of the measuring apparatus 1A. Further, the memory part 71 stores the pulse wave signal detected by the measuring unit 4 and the pulse rate analyzed by the analysis part 72.

The analysis part 72 analyzes the pulse wave signal input from the measuring unit 4 and determines the pulse rate. Specifically, the analysis part 72 performs a frequency analysis of FFT (Fast Fourier Transform) or the like on the pulse wave signal, extracts the frequency of the pulse from the obtained analysis result (power spectrum), and calculates the pulse rate based on the frequency of the pulse. Note that the analysis part 72 may calculate the pulse rate using another method, not limited to the calculation of the pulse rate.

### Configuration of Sensor

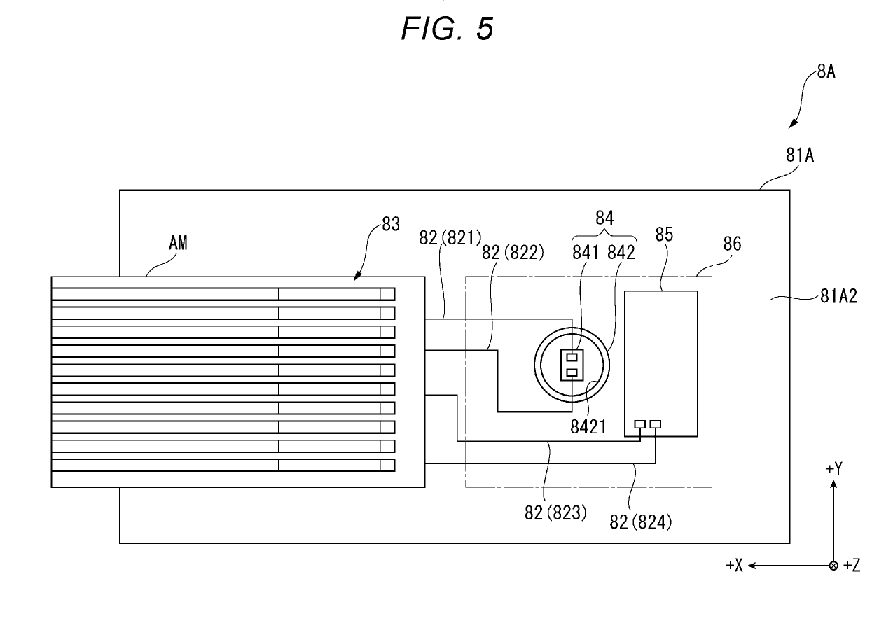

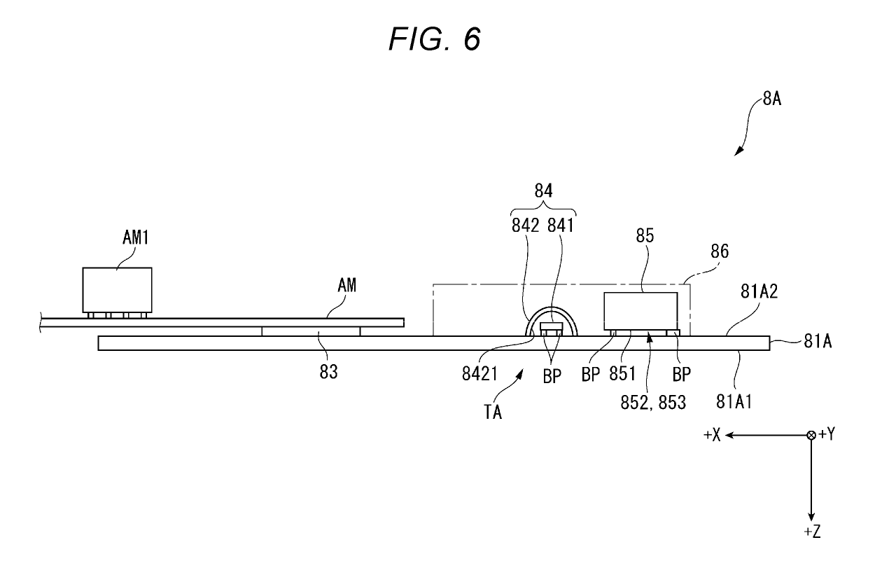

FIG. 5 shows the sensor 8A as seen from an opposite side to a light-exiting side, in other words, the sensor 8A as seen from inside of the housing 2. Further, FIG. 6 shows the sensor 8A as seen from a side.

The sensor 8A corresponds to a biological information detection sensor according to the invention. The sensor 8A is a photoelectric sensor (reflection photoelectric sensor) that emits light (detection light e.g. green light) to the body of the attached part US1 and detects the light reflected by the body. Specifically, the sensor 8A detects an intensity change of the detection light as biological information, pulse wave, and outputs the pulse wave signal (detection signal) indicating the detected pulse wave to the processing unit 7 via a connecting part (connector) AM.

As shown in FIGS. 5 and 6, the sensor 8A includes a optically transparent member 81A, and electrode patterns 82, an electrical circuit 83, the light emitting part (light emitter) 84, the light receiving part (light receiver) 85, and a sealing member 86 respectively located on the optically transparent member 81A.

### Configuration of Optically Transparent Member

The optically transparent member 81A contacts with the body surface (e.g. the body surface of the wrist) part of a user US1. The optically transparent member 81A is exposed on the back side of the housing 2 (i.e., on the attached part US1 side when the measuring apparatus 1A is attached) via the opening 222 of the housing (see FIG. 3), and the surface on the side in the +Z-direction exposed outside of the housing 2 in the optically transparent member 81A is a contact surface 81A1 (first surface) to be in contact with the body surface. In other words, the opening portion 222 of the housing 2 is closed by the optically transparent member 81A and the opening portion 222 of the housing 2 (specifically, the inside area of the opening portion 222) and the contact surface 81A1 overlap in the plan view from the side in the +Z-direction.

In the optically transparent member 81A, a surface opposed to the contact surface 81A1 (an opposite surface to the contact surface 81A1) is a mount surface 81A2 (second surface) on which the electrode patterns 82, the electrical circuit 83, the light emitting part 84, and the light receiving part 85 are provided.

Further, the optically transparent member 81A is formed using a light-transmissive material through which light can pass. Specifically, the optically transparent member 81A is a optically transparent member that can transmit the light emitted from the light emitting part 84 and the light entered into the light receiving part 85. That is, a passage area TA (FIG. 6) through which light can pass is provided in the optically transparent member 81A.

Note that, in the following description, an example using a glass substrate as the optically transparent member 81A will be explained. However, the optically transparent member 81A is not limited to that, but may be another optically transparent member of a resin substrate formed using a transparent resin or sapphire substrate.

### Configurations of Electrode Patterns and Electrical Circuit

A plurality of the electrode patterns 82 are transparent electrodes of transparent conducting films formed on the mount surface 81A2. Specifically, the electrode patterns 82 include electrode patterns 821, 822 with one ends connected to the electrical circuit 83 and the other ends connected to a light emitting device 841 of the light emitting part 84, and electrode patterns 823, 824 with one ends connected to the electrical circuit 83 and the other ends connected to the light receiving part 85. Note that the electrode patterns 82 are not limited to the transparent electrodes, but may be metal films formed by pattern printing or evaporation.

The electrical circuit 83 is a part to which an end portion of the connecting part AM is connected. The electrical circuit 83 is formed using an anisotropic conductive film (AFC) placed in contact with the respective electrode patterns 821 to 824. By the electrical circuit 83, the respective electrode patterns 821 to 824 and the connecting part AM are electrically connected.

Note that, in the embodiment, the connecting part AM is formed using flexible printed circuits (FPC), and an analog circuit AM1 (see FIG. 6) as an AFE (Analog Front End) is mounted on the connecting part AM. The analog circuit AM1 has e.g. an amplifier, an A/D converter, and a filter, and outputs a detection signal via the elements to the processing unit 7. The connecting part AM and the sensor 8A may be collectively referred to as a sensor module connected to the processing unit 7 as the circuit board.

### Configuration of Light Emitting Part

The light emitting part 84 irradiates the contacted part with detection light, is placed on the mount surface 81A2, and emits light toward the contact surface 81A1 side via the optically transparent member 81A. The light emitting part 84 has the light emitting device 841 and a reflection member (reflector) 842.

The light emitting device 841 is fixed by a bump BP (e.g. gold bump or solder bump) placed on the electrode patterns 821 and 822 with the light emission surface toward the mount surface 81A2, and emits green light toward the mount surface 81A2 by the current supplied via the electrode patterns 821 and 822. Accordingly, the light emitted by the light emitting device 841 passes through the passage area TA of the optically transparent member 81A and is output from the contact surface 81A1 of the optically transparent member 81A.

Note that, in the embodiment, the light emitting device 841 is an LED device, however, may be another light emitting device such as an organic EL device.

The reflection member 842 covers the light emitting device 841 on the mount surface 81A2, reflects light not traveling to the body of the user US (e.g. the light emitted toward the side in the −Z-direction) of the lights emitted from the light emitting device 841, and outputs the light toward the contact surface 81A1 side (toward the side in the +Z-direction) of the optically transparent member 81A via the optically transparent member 81A. The reflection member 842 is formed in a concave curved shape with one end open, and a concave curved surface 8421 inside is a reflection surface. The opening end is fixed to the mount surface 81A2 with the reflection member 842 placed on the mount surface 81A2 to cover the light emitting device 841 when the optically transparent member 81A is seen from the mount surface 81A2 side (the side in the −Z-direction). That is, as shown in FIG. 5, as seen along the +Z-direction from the mount surface 81A2 toward the contact surface 81A1 (in other words, as seen from the side in the −Z-direction), the concave curved surface 8421 of the reflection member 842 overlaps with the light emitting device 841. Further, as shown in FIG. 6, in the sectional view as seen along the +Y-direction as the direction orthogonal to the +Z-direction (in other words, when the section along the XZ-plane is seen), the light emitting device 841 is surrounded by the concave curved surface 8421 and the mount surface 81A2.

Note that, in the embodiment, at least the reflection surface of the reflection member 842 is formed using a metal. Accordingly, when the reflection member 842 and the electrode patterns 821 and 822 come into contact, short circuit may occur. Concave portions (not shown) for avoiding the electrode patterns 821 and 822 are formed in the opening end of the reflection member 842, and thereby, the contact between the reflection member 842 and the electrode patterns 821 and 822 is suppressed.

Here, the passage area TA includes an area of the optically transparent member 81A through which the light emitted by the light emitting part 84 passes, i.e., an area in which the light emitting part 84 and the contact surface 81A1 or mount surface 81A2 overlap in the plan view from the side in the +Z-direction.

### Configuration of Light Receiving Part

The light receiving part 85 receives the light emitted from the light emitting part 84 and reflected by the body of the user US (reflected light) and outputs the pulse wave signal as a signal of the voltage according to the intensity of the received light. The light receiving part 85 is fixed to the mount surface 81A2 by the bump BP like the light emitting part 84 with a detection surface 851 that detects the light toward the mount surface 81A2.

As described above, the light emitting part 84 and the light receiving part 85 are mounted face-down on the mount surface 81A2 of the optically transparent member 81A as the substrate with the electrode patterns 82 formed thereon.

The light receiving part 85 has a color filter 852 and an angle limiting filter 853 that cover the detection surface 851.

The color filter 852 is a filter that transmits light in the same wavelength range as that of the light emitted by the light emitting part 84, but does not transmit lights having wavelengths in other wavelength ranges. The color filter 852 is provided, and thereby, entry of other light (e.g. outside light) than the reflected light into the detection surface 851 and reduction in detection accuracy of the detection light are suppressed.

The angle limiting filter 853 is a filter that transmits light at an incident angle (an angle of incident light relative to the normal of the detection surface 851) equal to or smaller than a predetermined value (e.g. 30°) and suppresses transmission of lights at incident angles larger than the predetermined value. The angle limiting filter 853 is provided, and thereby, entry of the light emitted from the light emitting part 84 directly from the light emitting part 84 into the light receiving part 85 is suppressed.

### Configuration of Sealing Member

The sealing member 86 is provided on the mount surface 81A2 to cover the light emitting part 84 and the light receiving part 85 and has a function of protecting the light emitting part 84 and the light receiving part 85. Specifically, the sealing member 86 is formed by a sealing resin provided to cover the light emitting part 84 and the light receiving part 85 placed on the mount surface 81A2.

Note that it is preferable that the sealing resin does not exist inside of the reflection member 842 having the concave curved shape forming the light emitting part 84, however, when the sealing resin is transparent, the sealing resin may fill inside of the reflection member 842. On the other hand, as the light emitting part 84, a part filled with a resin between the light emitting device 841 and the inner surface of the reflection member 842 in advance may be employed.

### Advantages of First Embodiment

According to the biological information measuring apparatus 1A of the above described embodiment, the following advantages may be offered.

The biological information measuring apparatus 1A includes the housing 2 having the back part 22 as the contact part to be in contact with the body of the user US, the light emitting part 84 that emits light toward the body of the user US, the light receiving part 85 that receives the reflected light reflected by the body of the user US, the processing unit 7 that processes the signals from the light receiving part 85 and determines the pulse wave and the pulse rate as the biological information, and the optically transparent member 81A placed in the back part 22 and transmits the light emitted by the light emitting part 84 and the light entered into the light receiving part 85. The light emitting part 84 and the light receiving part 85 are placed on the mount surface 81A2 of the optically transparent member 81A. Thereby, compared to the case where the substrate on which the light emitting part and the light receiving part are placed on the surface toward the side in the +Z-direction is placed within the housing, the light emitting part 84 and the light receiving part 85 may be protected, and further, the distances between the optically transparent member 81A and the light emitting part 84 and light receiving part 85 may be made shorter. Accordingly, attenuation of the light emitted from the light emitting part 84 to the body of the user US and attenuation of the light reflected by the body of the user US and received by the light receiving part 85 may be suppressed. Therefore, detection and measurement accuracy of the pulse wave and the pulse rate by the biological information measuring apparatus 1A may be improved and precision of the pulse wave and the pulse rate may be improved. Further, the distances between the optically transparent member 81A and the light emitting part 84 and light receiving part 85 may be made shorter, and thereby, the sensor 8A may be made thinner and the biological information measuring apparatus 1A may be made thinner.

The optically transparent member 81A has the contact surface 81A1 as the first surface that can be in contact with the body of the user US, and the mount surface 81A2 as the second surface opposed to the contact surface 81A1, on which the light emitting part 84 and the light receiving part 85 are placed. Thereby, exposure of the light emitting part 84 and the light receiving part 85 outside of the housing 2 may be reliably suppressed. Therefore, the light emitting part 84 and the light receiving part 85 may be protected. Further, the contact surface 81A1 can be in contact with the body of the user US, and thereby, the distance between the optically transparent member 81A and the body of the user US may be made shorter. Accordingly, the optical path length of the light reaching the body of the user US from the light emitting part 84 and the optical path length of the light reaching the light receiving part 85 from the body of the user US may be made shorter, and the above described attenuation of the lights may be further suppressed. Therefore, the measurement accuracy of the biological information (pulse wave and pulse rate) by the biological information measuring apparatus 1A may be further improved, and the precision of the measured biological information may be further improved.

The measuring apparatus 1A has the connecting part AM that connects the processing unit 7 and the mount surface 81A2. The mount surface 81A2 has the electrode patterns 82 that electrically connect the connecting part AM and the light emitting part 84 and light receiving part 85. Accordingly, electric power may be supplied to the light emitting part 84 and the detection signal may be transmitted from the light receiving part 85 using the electrode patterns 82 located on the mount surface 81A2. Thereby, compared to the case where the power is supplied and the signal is transmitted to the light emitting part 84 and the light receiving part 85 via cables, the configuration of the measuring apparatus 1A may be simplified. Therefore, assembly of the sensor 8A may be easily performed and the sensor 8A, i.e., the measuring apparatus 1A may be made thinner. Further, the light emitting part 84 and light receiving part 85 and the processing unit 7 may be reliably connected via the connecting part AM connected to the electrode patterns 82.

The light emitting part 84 has the light emitting device 841 that emits light and the reflection member 842 that covers the light emitting device 841 and has the concave curved surface 8421 reflecting the light entering from the light emitting device 841 toward the body of the user US. Thereby, the light generated in the light emitting device 841 may be effectively radiated to the body of the user US. Therefore, use efficiency of the light generated in the light emitting device 841 may be improved. Further, the reflection member 842 covers the light emitting device 841, and thereby, entry of part of the light emitted from the light emitting device 841 into a component within the housing 2 may be suppressed. Therefore, deterioration of components due to entry of unnecessary light may be suppressed.

The optically transparent member 81A is the glass substrate. Thereby, the glass substrate generally has higher strength and may easily maintain the shape of the optically transparent member 81A to be in contact with the body of the user US. Further, the glass substrate is generally inexpensive, and rise of the manufacturing cost of the sensor 8A, i.e., the measuring apparatus 1A may be suppressed.

### Second Embodiment

Next, the second embodiment of the invention will be explained.

A biological information measuring apparatus according to the embodiment has the similar configuration to the biological information measuring apparatus 1A shown in the first embodiment, but differs in the configuration of the optically transparent member. Note that, in the following explanation, the same or substantially the same parts as parts that have been already explained will have the same signs and their explanation will be omitted.

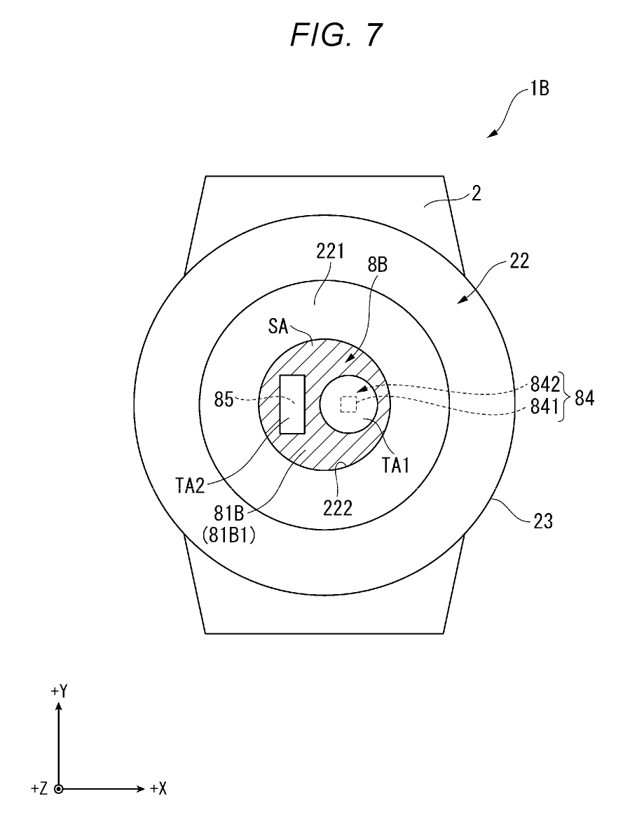

FIG. 7 shows a sensor 8B of a biological information measuring apparatus 1B according to the embodiment as seen from a light-exiting side (back side). In FIG. 7, a shield area SA, which will be described later, is hatched.

The biological information measuring apparatus 1B according to the embodiment has the same configuration and function as the above described biological information measuring apparatus 1A except that the apparatus has the sensor 8B as a biological information detection sensor in place of the sensor 8A. Further, as shown in FIG. 7, the sensor 8B has the same configuration and function as the above described sensor 8A except that the sensor has a optically transparent member 81B in place of the optically transparent member 81A. That is, the sensor 8B is connected to the processing unit 7 as a circuit board that processes the detection signal (pulse wave signal) by the sensor 8B via the connecting part AM connected to the electrical circuit 83 of the sensor 8B.

The optically transparent member 81B is formed using a glass substrate like the optically transparent member 81A exemplified in the first embodiment. The optically transparent member 81B has a contact surface 81B1 (first surface) to be in contact with the contacted part of the user US, and amount surface (not shown in FIG. 7) as a surface opposed to the contact surface 81B1 (second surface, an opposite surface to the contact surface 81B1). On the mount surface of the optically transparent member 81B, like the mount surface 81A2, the electrode patterns 82 and the electrical circuit 83 are formed, and the light emitting part 84 and the light receiving part 85 are provided to be electrically connected to the electrode patterns 82. The light emitting part 84 and the light receiving part 85 placed on the mount surface are sealed by the sealing member 86.

Here, the light emitted from the light emitting part 84 located on the mount surface of the optically transparent member 81B and the light reflected by the body of the user US can pass through the optically transparent member 81B. However, if the light can pass through the entire surface of the optically transparent member 81B, the user US can visually recognize inside of the housing 2 and the appearance of the measuring apparatus may be degraded.

On the other hand, in the biological information measuring apparatus 1B according to the embodiment, another area than a passage area TA1 through which the light emitted from the light emitting part 84 passes in the optically transparent member 81B and a passage area TA2 through which the light reflected by the body of the user US and entered into the light receiving part 85 passes is the shield area SA (the hatched area in FIG. 7) through which the light does not pass. Thereby, the sensor 8B is adapted so that the user may not visually recognize inside of the housing 2.

Note that the shield area SA may be formed, for example, by applying paint that shields light or forming a light-shielding film in the other area than the passage areas TA1, TA2 of the areas exposed from the opening portion 222 of the housing 2 in the contact surface 81B1.

Alternatively, a substrate formed using a non-light-transmissive material, in which opening portions according to the passage areas TA1, TA2 are formed may be employed in place of the optically transparent member 81B. In this case, light-transmissive materials may be placed within the respective opening portions.

### Advantages of Second Embodiment

The biological information measuring apparatus 1B according to the above described embodiment may offer not only the same advantages as the biological information measuring apparatus 1A but also the following advantage.

The optically transparent member 81B has the shield area SA for shielding light provided in the position except the respective passage area TA1 through which the light emitted from the light emitting part 84 passes and passage area TA2 of the light entered into the light receiving part 85. Thereby, observation inside of the housing 2 may be suppressed by the shield area SA. Therefore, the appearance of the biological information measuring apparatus 1B may be made better.

### Third Embodiment

Next, the third embodiment of the invention will be explained.

A biological information measuring apparatus according to the embodiment has the similar configuration to the biological information measuring apparatus 1A shown in the first embodiment, but differs from the biological information measuring apparatus 1A in that the optically transparent member has a convex portion and a concave portion that function as lenses. Note that, in the following explanation, the same or substantially the same parts as parts that have been already explained will have the same signs and their explanation will be omitted.

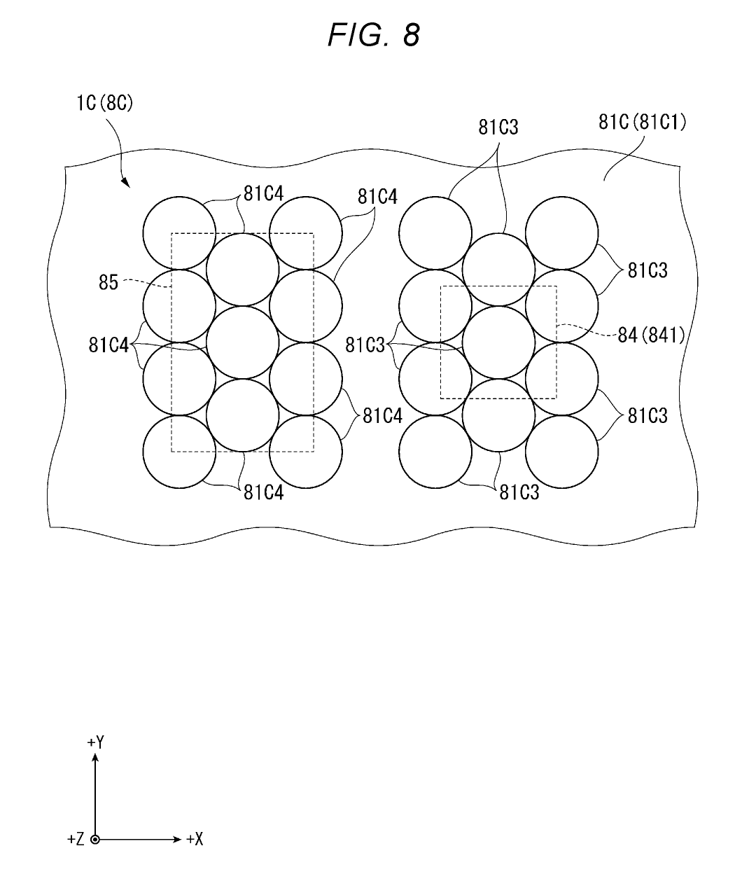

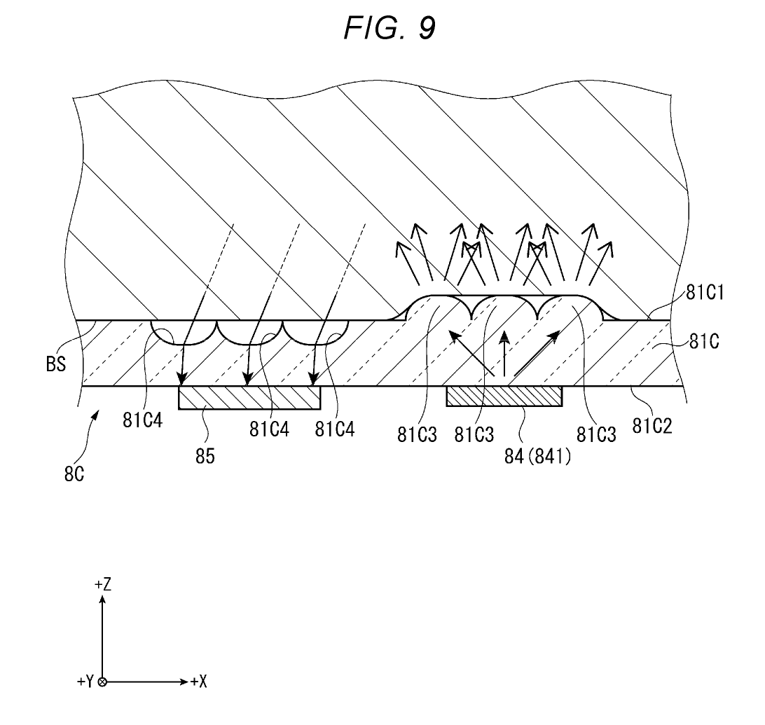

FIG. 8 is a schematic diagram showing a part of a sensor 8C of a biological information measuring apparatus 1C according to the embodiment as seen from the light-exiting side (the side in the +Z-direction), and FIG. 9 is a schematic diagram showing optical paths of lights emitted from the light emitting part 84 and optical paths of the lights received by the light receiving part 85 of the sensor 8C.

The biological information measuring apparatus 1C according to the embodiment has the same configuration and function as the above described biological information measuring apparatus 1A except that the apparatus has the sensor 8C as a biological information detection sensor in place of the sensor 8A. Further, as shown in FIGS. 8 and 9, the sensor 8C has the same configuration as the above described sensor 8A except that the sensor has a optically transparent member 81C in place of the optically transparent member 81A. That is, the sensor 8C is connected to the processing unit 7 as a circuit board that processes the detection signal (pulse wave signal) by the sensor 8C via the connecting part AM connected to the electrical circuit 83 of the sensor 8C.

The optically transparent member 81C is formed using a glass substrate like the optically transparent member 81A, and to be in contact with the contacted part of the user US. That is, the optically transparent member 81C is a contact member to be in contact with the body of the user. The optically transparent member 81C has a contact surface 81C1 (first surface) that is exposed outside of the housing 2 and can be in contact with the body of the user, and a mount surface 81C2 (second surface) as an opposite surface to the contact surface 81C1.

Of the surfaces, on the mount surface 81C2, like the mount surface 81A2 of the optically transparent member 81A, the electrode patterns 82 are formed, and additionally, the electrical circuit 83, the light emitting part 84, and the light receiving part 85 are placed. Further, on the mount surface 81C2, the sealing member 86 (not shown) that covers the light emitting part 84 and the light receiving part 85 is formed. Note that, in FIGS. 8 and 9, the reflection member 842 forming the light emitting part 84 is not shown.

The contact surface 81C1 comes into contact with a body surface BS (see FIG. 9) of the user like the contact surfaces 81A1, 81B1. In the contact surface 81C1, a plurality of convex portions 81C3 and a plurality of concave portions 81C4 are provided.

As shown in FIG. 8, the plurality of convex portions 81C3 are placed in the passage area TA through which the light emitted from the light emitting part 84 passes in the optically transparent member 81C. Specifically, the plurality of convex portions 81C3 are placed adjacent to each other in the positions overlapping with the light emitting device 841 as seen along the −Z direction as the direction from the contact surface 81C1 toward the mount surface 81C2 of the sensor 8C. That is, the optically transparent member 81C has the convex portions 81C3 located in the passage area TA of the contact surface 81C1 and overlapping with the light emitting part 84 as seen from the side in the +Z-direction.

As shown in FIG. 9, these convex portions 81C3 project in the +Z-direction (the direction from the mount surface 81C2 as the second surface toward the contact surface 81C1 as the first surface), and respectively function as convex lenses. Further, each convex portion 81C3 diffuses the light entered from the light emitting part 84 (light emitting device 841). In other words, each convex portion 81C3 diffusionally emits the light entered from the light emitting part 84 toward the body surface BS as a part of the body of the user US.

Note that FIGS. 8 and 9 show the example in which the plurality of convex portions 81C3 are placed in correspondence with the light emitting device 841, however, the plurality of convex portions 81C3 or the single convex portion 81C3 may be provided in the position overlapping with the light emitting device 841 and the reflection member 842 as seen from the side in the +Z-direction. That is, in the contact surface 81C1, it is only necessary that at least one convex portion 81C3 is provided within the area through which the light emitted from the light emitting part 84 passes.

As shown in FIG. 8, the plurality of concave portions 81C4 are placed in the passage are TA through which the light entered into the light receiving part 85 from the side in the +Z-direction passes in the optically transparent member 81C. Specifically, the plurality of concave portions 81C4 are placed adjacent to each other in the positions overlapping with the light receiving part 85 as seen along the −Z direction as the direction from the contact surface 81C1 toward the mount surface 81C2 of the sensor 8C. That is, the optically transparent member 81C has the concave portions 81C4 located in the passage area TA of the contact surface 81C1 and overlapping with the light receiving part 85 as seen from the side in the +Z-direction.

As shown in FIG. 9, these concave portions 81C4 are recessed in the −Z-direction (the direction from the contact surface 81C1 as the first surface toward the mount surface 81C2 as the second surface), and respectively function as concave lenses. Further, each concave portion 81C4 collects the light reflected by the body of the user US in the light receiving part 85.

Note that FIGS. 8 and 9 show the example in which the plurality of concave portions 81C4 are placed in correspondence with the light receiving part 85, however, the single concave portion 81C4 may be provided in a position overlapping with the light receiving part 85 as seen from the side in the +Z-direction. That is, in the contact surface 81C1, it is only necessary that at least one concave portion 81C4 is provided within the area through which the light entered into the light receiving part 85 passes.

In the embodiment, the plurality of convex portions 81C3 and the plurality of concave portions 81C4 are respectively integrally formed with the optically transparent member 81C. However, the portions are not limited to that. For example, the convex portions 81C3 and the concave portions 81C4 may be provided in the optically transparent member 81C by placement of a sheet with convex portions and concave portions formed therein on the contact surface 81C1 or the like.

### Advantages of Third Embodiment

The biological information measuring apparatus 1C according to the above described embodiment may offer not only the same advantages as the biological information measuring apparatus 1A but also the following advantage.

The optically transparent member 81C forming the sensor 8C has the convex portions 81C3 that project in the +Z-direction as the direction from the mount surface 81C2 (second surface) toward the contact surface 81C1 (first surface) and overlap with the light emitting part 84 as seen along the −Z-direction as the direction from the contact surface 81C1 toward the mount surface 81C2. Thereby, the convex portions 81C3 may be allowed to function as convex lenses that act on the light emitted from the light emitting part 84. Accordingly, the light emitted from the light emitting part 84 may be diffused by the lens effect of the convex portions 81C3. Therefore, the light emitted from the light emitting part 84 may be efficiently output to the body of the user US and the measurement accuracy of the pulse wave and the pulse rate as the biological information may be improved.

The optically transparent member 81C forming the sensor 8C has the concave portions 81C4 recessed in the direction from the contact surface 81C1 (first surface) toward the mount surface 81C2 (second surface) and overlapping with the light receiving part 85 as seen along the −Z-direction as the direction from the contact surface 81C1 toward the mount surface 81C2. Thereby, the concave portions 81C4 may be allowed to function as concave lenses that act on the light entered into the light receiving part 85. Accordingly, the light reflected by the body of the user US and diffused may be collected in the light receiving part 85 by the lens effect of the concave portions 81C4. Therefore, the light may be efficiently entered into the light receiving part 85 and the measurement accuracy of the pulse wave and the pulse rate as the biological information may be improved.

Further, the convex portions 81C3 and the concave portions 81C4 are located in the contact surface 81C1 to be into contact with the body of the user US in the optically transparent member 81C. Thereby, drainage of sweat or the like of the optically transparent member 81C to be in contact with the body surface BS may be improved.

Here, in the case where only one convex portion 81C3 is provided to overlap with the whole light emitting part 84 as seen from the side in the +Z-direction, the amount of projection of the convex portion 81C3 becomes larger and the thickness dimension of the optically transparent member becomes larger. Further, in the case where only one concave portion 81C4 is provided to overlap with the whole light receiving part 85 as seen from the side in the +Z-direction, the amount of the recess of the concave portion 81C4 becomes larger and, to secure strength, it is necessary to increase the thickness dimension of the optically transparent member.

On the other hand, in the embodiment, the plurality of convex portions 81C3 respectively overlapping with the light emitting part 84 as seen from the side in the +Z-direction and the plurality of concave portions 81C4 respectively overlapping with the light receiving part 85 are provided in the optically transparent member 81C. Thereby, the amount of the projection of each convex portion 81C3 and the the amount of the recess of each concave portion 81C4 may be made smaller. Therefore, compared to the case where respective one convex portion 81C3 and concave portion 81C4 are provided, increase of the thickness dimension of the optically transparent member 81C may be suppressed.

### Modifications of Embodiments

The invention is not limited to the above described respective embodiments, but the invention includes modifications, improvements, etc. within the range in which the purpose of the invention may be achieved.

In the above described respective embodiments, the biological information measuring apparatuses 1A, 1B, 1C are explained as wearable apparatuses attached to the attached part US1 such as a wrist of the user US and used. However, the attachment part of the biological information measuring apparatus is not limited to the wrist, but may be another part. Further, the sensors 8A, 8B, 8C are not limited to the employment for the biological information measuring apparatuses 1A, 1B, 1C dedicated to the function of measuring biological information, but may have e.g. configurations provided in a wristwatch, band, or the like to output or transmit detection results to an external apparatus. In this regard, the detection results may be transmitted to the external apparatus in wireless or wired connection.

In the above described respective embodiments, the sensors 8A, 8B, 8C have the single light emitting parts 84 and the single light receiving parts 85. However, the sensor is not limited to that, but may have one light emitting part 84 and a plurality of light receiving parts 85, or a plurality of light emitting parts 84 and one light receiving part 85. Further, the sensor may have a plurality of sets of at least one light emitting part 84 and at least one light receiving part 85.

In the above described respective embodiments, the light emitting part 84 is placed on the side in the +X-direction with respect to the light receiving part 85. However, the positions of the light emitting part 84 and the light receiving part 85 are not limited to those, but may be respectively appropriately changed. For example, the light emitting part 84 may be placed on the side in the −X-direction, on the side in the +Y-direction, or on the side in the −Y-direction with respect to the light receiving part 85. Further, the positions of the light emitting part 84 and the light receiving part 85 within the opening portion 222 formed in the back part 22 may be respectively appropriately changed. For example, one of the light emitting part 84 and the light receiving part 85 may be placed at the center of the opening portion 222.

In the above described respective embodiments, the connecting parts AM connecting the sensors 8A, 8B, 8C and the processing units 7 as the circuit boards are the flexible boards. However, the biological information measuring apparatus is not limited to that, but may have a configuration in which the sensor and the processing unit are connected by a cable or connector or a rigid board. That is, the connection form between the sensor and the processing unit is not limited to that described as above.

In the above described respective embodiments, the electrode patterns 82 connecting the electrical circuits 83 and the light emitting parts 84 and light receiving parts 85 are formed on the respective mount surfaces (second surfaces) of the optically transparent members 81A, 81B, 81C. However, the power supply lines and the signal transmission lines connected to the connecting parts AM are not limited to the electrode patterns formed on the mount surface as long as electric power may be supplied to the light emitting part 84 and the detection signal may be transmitted from the light receiving part 85. For example, the electrical circuit 83 and the light emitting part 84 and light receiving part 85 may be connected by electric wires or the like. That is, the connection form between the electrical circuit 83 and the light emitting part 84 and light receiving part 85 is not limited to those described as above.

In the above described respective embodiments, the light emitting part 84 has the light emitting device 841 that emits light and the reflection member 842 that covers the light emitting device 841 as seen from the side in the −Z-direction and reflects the light entered from the light emitting device 841 toward the body of the user US. However, the part is not limited to that, but may have no reflection member 842. Alternatively, a light-shielding part that shields the light emitted from the light emitting device 841 and suppresses direct entry of the light emitted from the light emitting device 841 into the light receiving part 85 may be provided between the light emitting device 841 and the light receiving part 85. Further, the configuration of the reflection member is not limited to the reflection member 842 having the concave curved surface, but may be appropriately changed. For example, as the reflection member, a resin film or metal film covering the light emitting device 841 or a white or silver mold member may be employed. As the colors of the respective films and the mold members in this case, white or silver may be exemplified for efficient reflection of incident light.

In the above described respective embodiments, the optically transparent members 81A, 81B, 81C are the glass substrates. However, the members are not limited to those, but may be other optically transparent members such as sapphire substrates or resin substrates.

Further, the contact surfaces 81A1, 81B1, 81C1 as the first surfaces in the respective optically transparent members 81A, 81B, 81C do not necessarily come into direct contact with the body of the user US, but may come into contact with the body of the user US via sheets provided on the contact surfaces or layer structures formed on the contact surfaces. That is, it is only necessary that the optically transparent member is provided to face the body of the user US when the sensor detects or measures the biological information.

Furthermore, the contact surface as the first surface in the optically transparent member is not necessarily flat, but may be formed in a curved shape that can press the body of the user US. For example, the contact surface may be a convex curved surface toward the body of the user US, i.e., a convex curved surface projecting in the direction in which the light emitted from the light emitting part travels. Similarly, the mount surface as the second surface in the optically transparent member is not necessarily flat. Moreover, the optically transparent member (contact surface) does not necessarily come into contact with the body of the user US.

In the above described respective embodiments, the light emitting parts 84 and the light receiving parts 85 are mounted on the mount surfaces of the optically transparent members 81A, 81B, 81C using the bumps BP. However, the mount form of the light emitting part 84 and the light receiving part 85 on the mount surface is not limited to that described as above.

The sensors 8A, 8B, 8C have the sealing members 86 formed using the sealing resins and covering the light emitting parts 84 and the light receiving parts 85. However, the sensor is not limited to that, but may have no sealing member 86. Note that, with the sealing members 86, strengths of the sensors 8A, 8B, 8C (strengths of the optically transparent members 81A, 81B, 81C) may be made higher and the optically transparent members 81A, 81B, 81C may be made thinner. In addition, total reflection of the light entered into the light receiving part 85 may be suppressed, and thus, the amount of light received by the light receiving part 85 may be increased.

In the above described respective embodiments, the analog circuits AM1 as AFEs are mounted on the connecting parts AM. However, the analog circuits AM1 are not limited to those, but, for example, may be provided on or supported by the mount surfaces (second surfaces) of the optically transparent members 81A, 81B, 81C or provided on or supported by the processing units 7 as the processing boards. That is, the analog circuits AM1 may be included in the sensors 8A, 8B, 8C or not.

In the above described first embodiment, the contact surface 81A1 as the first surface of the optically transparent member 81A in the sensor 8A is nearly flat and the passage area TA is provided on the entire optically transparent member 81A. In the above described second embodiment, the passage areas TA1, TA2 are provided in correspondence with the light emitting part 84 and the light receiving part 85 on the contact surface 81B1 as the first surface of the optically transparent member 81B in the sensor 8B, and the area except the passage areas TA1, TA2 is the shield area SA. In the above described third embodiment, the contact surface 81C1 as the first surface of the optically transparent member 81C in the sensor 8C has at least one convex portion 81C3 overlapping with the light emitting part 84 as seen from the side in the +Z-direction and at least one concave portion 81C4 overlapping with the light receiving part 85 as seen from the side in the +Z-direction. The configurations shown in these embodiments may be combined with one another. For example, at least one convex portion 81C3 or concave portion may be placed in the passage area TA1 of the optically transparent member 81B, at least one concave portion 81C4 or convex portion is placed in the passage area TA2, and the other area than the passage areas TA1, TA2 may be the shield area SA. Alternatively, as will be described later, the convex portion or concave portion placed in the passage areas TA1, TA2 are not limited to be located on the contact surface of the optically transparent member, but may be located on the mount surface as the second surface.

In the above described third embodiment, the convex portion 81C3 diffuses and outputs the light entered from the light emitting part 84 and the concave portion 81C4 collects the light reflected by the body of the user US in the light receiving part 85. However, the portions are not limited to those, but at least one concave portion 81C4 overlapping with the light emitting part 84 may be placed and at least one convex portion 81C3 overlapping with the light receiving part 85 may be placed as seen from the side in the +Z-direction. Further, in the passage areas TA, TA1, TA2, a convex portion or concave portion overlapping with the light emitting part 84 may be placed and a convex portion or concave portion overlapping with the light receiving part 85 may be placed on the mount surface of the optically transparent member. Alternatively, none of a convex portion and a concave portion is placed on one of the light emitting part 84 and the light receiving part 85. In the case where a concave portion overlapping with the light emitting part 84 as seen from the side in the +Z-direction is placed, the light may be collected on the body of the user US.

In the above described respective embodiments, the sensors 8A, 8B, 8C detect the pulse wave as one kind of the biological information and the processing units 7 determine and analyze the pulse rates as another kind of the biological information based on the detection signals (pulse wave signals) by the sensors 8A, 8B, 8C. That is, the biological information measuring apparatuses 1A, 1B, 1C measure the pulse wave and the pulse rates. However, the biological information that can be measured by the biological information measuring apparatus according to the invention is not limited to that. For example, the biological information measuring apparatus may measure an amount of activity, calorie consumption, the maximum oxygen intake (VO₂max) based on the detection results by the sensor.

Further, the biological information measuring apparatus may include a motion sensor such as an acceleration sensor that can detect body motion information of the user or a position sensor (e.g. GPS sensor) that can measure position information.

What is claimed is:

1. A biological information measuring apparatus comprising:
a housing configured to have a contact part, the contact part contacts with a body of a user;
a light emitter configured to emit light toward the body of the user;
a light receiver configured to receive reflected light reflected by the body of the user;
a processor configured to process a signal from the light receiving part and determines biological information; and
a optically transparent member configured to be arranged to the contact part and transmitting the light from the light emitter and the reflected light, on which the light emitter and the light receiver are arranged.

2. The biological information measuring apparatus according to claim 1,
wherein the optically transparent member has:
a first surface that contacts with the body of the user; and
a second surface on which the light emitting part and the light receiving part are arranged.

3. The biological information measuring apparatus according to claim 2,
wherein the optically transparent member has a convex portion projecting in a first direction from the second surface toward the first surface, placed on the first surface, and overlapping with the light emitter in a plan view from the first direction.

4. The biological information measuring apparatus according to claim 2,
wherein the optically transparent member has a concave portion recessed in a second direction from the first surface toward the second surface, placed on the first surface, and overlapping with the light receiving part in a plan view from the second direction.

5. The biological information measuring apparatus according to claim 3,
wherein the optically transparent member has a concave portion recessed in a second direction from the first surface toward the second surface, placed on the first surface, and overlapping with the light receiving part in a plan view from the second direction.

6. The biological information measuring apparatus according to claim 2, further comprising:
an electrode pattern provided on the second surface and electrically connecting to the light emitter and the light receiver; and
a connector that electrically connects the electrode pattern and the processing unit.

7. The biological information measuring apparatus according to claim 3, further comprising:
an electrode pattern provided on the second surface and electrically connecting to the light emitter and the light receiver; and
a connector that electrically connects the electrode pattern and the processing unit.

8. The biological information measuring apparatus according to claim 4, further comprising:
an electrode pattern provided on the second surface and electrically connecting to the light emitter and the light receiver; and
a connector that electrically connects the electrode pattern and the processing unit.

9. The biological information measuring apparatus according to claim 1,
wherein the light emitter includes:
a light emitting device that emits light; and
a reflection member that covers the light emitting device and has a concave curved surface for reflecting the light entered from the light emitting device toward the body of the user.

10. The biological information measuring apparatus according to claim 2,
wherein the light emitter includes:
a light emitting device that emits light; and
a reflector that covers the light emitting device and has a concave curved surface for reflecting the light entered from the light emitting device toward the body of the user.

11. The biological information measuring apparatus according to claim 3,
wherein the light emitter includes:
a light emitting device that emits light; and
a reflector that covers the light emitting device and has a concave curved surface for reflecting the light entered from the light emitting device toward the body of the user.

12. The biological information measuring apparatus according to claim 1,
wherein the optically transparent member is a glass substrate or resin that transmits the light from the light emitter and the reflected light.

13. The biological information measuring apparatus according to claim 2,
wherein the optically transparent member is a glass substrate or resin that transmits the light from the light emitter and the reflected light.

14. The biological information measuring apparatus according to claim 3,
wherein the optically transparent member is a glass substrate or resin that transmits the reflected light.

15. The biological information measuring apparatus according to claim 1,
wherein the optically transparent member has a shield area that shields light in a position except a passage area for the light emitted from the light emitter and the reflected light.

16. The biological information measuring apparatus according to claim 2,
wherein the optically transparent member has a shield area that shields light in a position except a passage area for the light emitted from the light emitter and the reflected light.

17. The biological information measuring apparatus according to claim 3,
wherein the optically transparent member has a shield area that shields light in a position except a passage area for the light emitted from the light emitter and the reflected light.

18. A biological information detection sensor used for a biological information measuring apparatus that measures biological information of a user, comprising:
an optically transparent member configured to have a first surface to be in contact with a body of the user, and a second surface as an opposite surface to the first surface;
a light emitter configured to be arranged on the second surface and to emit light along a first direction from the second surface toward the first surface; and
a light receiver configured to be arranged on the second surface and receiving light traveling along a second direction from the first surface toward the second surface,
wherein the optically transparent member has a passage area through which at least the light emitted from the light emitter and the light entering the light receiver pass, and a shield area provided in a position except the passage area and shielding light.

19. The biological information detection sensor according to claim 18, further comprising an electrode pattern configured to be arranged on the second surface and electrically connecting to the light emitter and the light receiver.

20. The biological information detection sensor according to claim 18,
wherein the optically transparent member is a glass substrate or resin that transmits the light from the light emitter and the light to be received by the light receiver.
